(19) United States
(12) Patent Application Publication
(10) Pub. No.: US 2021/0236487 A1
(43) Pub. Date: Aug. 5, 2021

(54) Methods and Compositions for Treatment of Cancer with PARP Inhibitors

(71) Applicant: Rush University Medical Center, Chicago, IL (US)

(72) Inventors: Jamille M. SHAMMO, Park Ridge, IL (US); Lydia USHA, Chicago, IL (US)

(73) Assignee: Rush University Medical Center, Chicago, IL (US)

(21) Appl. No.: 17/051,836

(22) PCT Filed: May 1, 2019
(86) PCT No.: PCT/US2019/030144

§ 371 Date: Oct. 30, 2020

Related U.S. Application Data
(60) Provisional application No. 62/665,176, filed on May 1, 2018.

(51) Int. Cl. A61K 31/501; A61K 31/519; A61K 45/06; A61P 35/00; A61K 31/55; A61K 31/4439; A61K 31/5025; A61K 31/502; A61K 31/407; A61K 31/496; A61K 31/5355; A61K 31/28; A61K 31/5517; G01N 33/82

(52) U.S. Cl. CPC A61K 31/501; G01N 2800/52; A61K 45/06; A61P 35/00; A61K 31/55; A61K 31/4439; A61K 31/5025; A61K 31/502; A61K 31/407; A61K 31/496; A61K 31/5355; A61K 31/28; A61K 31/5517; G01N 33/82; A61K 31/519

(57) ABSTRACT

Methods of treating cancer in a subject and compositions are provided. The methods include administering a therapeutically effective amount of a poly (ADP ribose) polymerase (PARP) inhibitor to the subject in need thereof and administering a therapeutically effective amount of a folic acid supplement to the subject. Also described herein are methods for identifying a subject having cancer and having an elevated folate receptor expression level relative to a folate receptor expression level in a subject free of cancer and administering a therapeutically effective amount of a PARP inhibitor to the subject having the elevated folate receptor level. Also described herein are compositions including a PARP inhibitor and a folic acid supplement.

### CROSS-REFERENCE TO RELATED APPLICATIONS

This application claims the benefit of U.S. Provisional Patent Application No. 62/665,176, filed May 1, 2018, the entire contents of which are hereby incorporated herein by reference.

### BACKGROUND

Olaparib (Lynparza™) is the first targeted drug FDA approved for advanced BRCA-deficient ovarian cancer. Olaparib inhibits poly (ADP-ribose) polymerase (PARP) enzymes which are involved in DNA repair. Olaparib induces synthetic lethality in BRCA1/2 deficient tumor cells through the accumulation of double-stranded DNA breaks which cannot be accurately repaired (homologous recombination deficiency) (Ledermann, 2012). The approval of olaparib was based on the results of Phase II trials in women with advanced ovarian cancer and germline BRCA1/2 mutations (gBRCA-mutated advanced ovarian cancer) who have had three or more previous lines of chemotherapy. These trials demonstrated an overall response rate (ORR) of 34% (ibid) and significantly prolonged progression-free survival. (Ledermann #1 and #2, 2012, 2014)

Women with gBRCA-mutated advanced ovarian cancer achieve the best progression free survival (PFS) on olaparib therapy. The PFS in ovarian cancer patients with wild-type BRCA mutations is modest. However, some ovarian cancer patients with wild-type BRCA derive significant benefit from the drug. Great strides have been made to identify these patients (Spriggs D., 2016). Preliminary data suggest that there are other molecular markers that potentially can predict a benefit from olaparib as well as other PARP inhibitors. These molecular markers are somatic BRCA1 and 2 mutations in cancer cells as determined by tumor genomic profiling, germline and somatic mutations in other genes leading to homologous recombination deficiency (HRD) in the tumor as determined by an HRD assay (Lheureux S., 2017).

One side effect of PARP inhibitors is hematologic toxicity. The hematologic toxicity can require a reduction in the amount of PARP inhibitor administered and in some instances a cessation of the PARP inhibitor therapy. What is needed are methods of treatment that allow for continued administration of the PARP inhibitor administration and methods of identifying subjects that will benefit from PARP inhibitor administration.

### BRIEF SUMMARY

One embodiment described herein is a method for treating cancer in a subject comprising administering a therapeutically effective amount of a poly (ADP ribose) polymerase (PARP) inhibitor to the subject in need thereof and administering a therapeutically effective amount of a folic acid supplement to the subject.

In one aspect, the PARP inhibitor is selected from the group consisting of olaparib, rucaparib, niraparib, talazoparib, veliparib, CEP8983, CEP9722, E7016, INO-1001, AZD2461, E7449, SC10914, BGB-290 or fluzoparib. In another aspect the PARP inhibitor is olaparib.

In another aspect the cancer is selected from the group consisting of breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors stomach cancer, urinary cancer, or uterine cancer.

In another aspect, the subject has a BRCA1 or BRCA2 mutation.

In another aspect, the method further comprises monitoring folic acid levels in the subject.

In yet another aspect, the amount of the folic acid supplement is 0.25 mg/day up to 6 mg/day.

In one aspect, the PARP inhibitor is co-administered with a chemotherapeutic agent or radiotherapy.

Another embodiment described herein is a method for treating cancer in a subject comprising identifying an elevated folate receptor expression level on cancer cells of the subject relative to a folate receptor expression level on cells in a subject free of cancer and administering a therapeutically effective amount of a PARP inhibitor to the subject having the elevated folate receptor level.

In one aspect, the method further comprises monitoring a folic acid level in the subject.

In another aspect, the method further comprises administering a therapeutically effective amount of a folic acid supplement to the subject.

In yet another aspect the amount of the folic acid supplement is 0.25 mg/day up to 6 mg/day.

In another aspect, the cancer is selected from the group consisting of breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors stomach cancer, urinary cancer, or uterine cancer.

In one aspect, the PARP inhibitor is selected from the group consisting of olaparib, rucaparib, niraparib, talazoparib, veliparib, CEP8983, CEP9722, E7016, INO-1001, AZD2461, E7449, SC10914, BGB-290 or fluzoparib.

Another embodiment described herein is a composition for the treatment of cancer comprising a PARP inhibitor and a folic acid supplement.

In one aspect, the PARP inhibitor is selected from the group consisting of olaparib, rucaparib, niraparib, talazoparib, veliparib, CEP8983, CEP9722, E7016, INO-1001, AZD2461, E7449, SC10914, BGB-290 or fluzoparib.

Another embodiment described herein is a method of identifying a subject having cancer that is likely to benefit from a PARP inhibitor therapy comprising identifying an elevated folate receptor expression level on cancer cells of the subject relative to a folate receptor expression level on cells in a subject free of cancer; wherein subjects having the elevated folate receptor expression level on cancer cells are likely to benefit from a PARP inhibitor therapy.

In one aspect, the method further comprises administering a therapeutically effective amount of a PARP inhibitor to the subject having the elevated folate receptor level.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

Methods of treating cancer in a subject are provided. In some embodiments, the methods include administering a therapeutically effective amount of a poly (ADP ribose) polymerase (PARP) inhibitor and a therapeutically effective amount of a folic acid supplement to a subject. In some embodiments, the methods include identifying a subject having cancer and having an elevated folate receptor expression level relative to a folate receptor expression level in a subject free of cancer, and administering a therapeutically effective amount of a PARP inhibitor to the subject having the elevated folate receptor level. A folic acid supplement may also be administered to subjects having an elevated folate receptor expression level.

“Treating”, “treat”, or “treatment” within the context of the instant invention, means an alleviation of symptoms associated with a disorder or disease, or halt of further progression or worsening of those symptoms, or prevention or prophylaxis of the disease or disorder. For example, within the context of this invention, successful treatment may include an alleviation of symptoms related to cancer and/or folic acid deficiency.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition (e.g., a cancer), or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The term “subject” or “patient” as used herein, refers to a mammal, preferably a human.

PARP Inhibitors

A “PARP inhibitor” refers to any agent that can inhibit the activity of Poly(ADP-ribose) polymerase. PARP is a member of a family of proteins that is involved in a number of cellular processes, such as DNA repair including single strand breaks, and programmed cell death. A PARP inhibitor reduces the functioning of a PARP. The PARP inhibitor may be selected from the group consisting of a small molecule, a nucleic acid, a nucleic acid analog or derivative, a peptide, a peptidomimetic, a protein, an antibody or an antigen-binding fragment thereof, a monosaccharide, a disaccharide, a trisaccharide, an oligosaccharide, a polysaccharide, a lipid, a glycosaminoglycan, an extract made from a biological material, and combinations thereof. By way of non-limiting example, the PARP inhibitor is selected from olaparib (AZD2281) (4-[(3-[(4-cyclopropylcarbonyl)piperazin-4-yl]carbonyl)-4-fluorophenyl]methyl(2H)-phthalazin-1-one), veliparib (2-((R)-2-methylpyrrolidin-2-yl)-1H-benzimidazole-4-carboxamide), CEP-8983 (11-methoxy-4,5,6,7-tetrahydro-1H-cyclopenta[a]pyrrolo[3,4-c]carbazole-1,3(2H)-dione) or a prodrug thereof (e.g. CEP-9722), rucaparib (8-Fluoro-2-{4-[(methylamino)methyl]phenyl}-1,3,4,5-tetrahydro-6H-azepino[5,4,3-cd]indol-6-one), E7016 (10-((4-Hydroxypiperidin-1-yl)methyl)chromeno-[4,3,2-de]phthalazin-3(2H)-one), talazoparib (BMN-673) ((8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one), niraparib (2-[4-[(3S)-3-Piperidyl]phenyl]indazole-7-carboxamide), INO-1001 (4-phenoxy-3-pyrrolidin-1-yl-5-sulfamoyl-benzoic acid), AZD2461 (4-[[4-fluoro-3-[(4-methoxy-1-piperidinyl)carbonyl]phenyl]methyl]-1(2H)-phthalazinone), E7449 (8-(isoindolin-2-ylmethyl)-2H-pyridazino[3,4,5-de]quinazolin-3(9H)-one), SC10914 (dichlorozirconium; di(inden-1-yl)-dimethylsilane), BGB-290 (Beigene, Cambridge, Mass.), and Fluzoparib, (Jiangsu Hengrui Medicine Co., Ltd., Jiangsu, China).

Folic Acid

Folic Acid (or folate) is one of the B vitamins. A folic acid deficiency can lead to anemia. Anemia in subjects receiving cancer therapies can lead to reduction or cessation of the therapy depending on the severity of the anemia. Patients receiving some types of cancer therapy can develop a folic acid deficiency. As described in more detail below, patients receiving PARP inhibitor therapy develop folic acid deficiency. In some embodiments described herein, subjects receiving a therapeutically effective dose of a PARP inhibitor will also receive a therapeutically effective dose of a folic acid supplement. The folic acid levels may be monitored during the PARP inhibitor administration and may be modified depending on the patient's response. By way of non-limiting example, a patient may receive 0.25 mg/day up to 6 mg/day of a folic acid supplement. Other amounts are possible and may be determined by the physician. The folic acid supplement may be administered in a single dose, in multiple doses and together or separate from a dose of a PARP inhibitor as described in more detail below.

Cancers and Other Diseases

As used herein, a “cancer” in a subject refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. The cancer cells may be in the form of a tumor; such cells may exist locally within a subject, or circulate in the blood stream as independent cells, for example, leukemic cells. A cancer can include, but is not limited to, breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors, stomach cancer, urinary cancer, and uterine cancer. In some aspects, the disease may be sensitive to anti-folates, such as choriocarcinoma and gestational trophoblastic disease.

Genetic Aberrations

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, for example by means of mutation or polymorphism in the encoding nucleic acid, or by means of amplification, mutation or polymorphism in a gene encoding a regulatory factor. However, the cancer is not limited to only BRACA1 and/or BRACA2 deficient cells. In some embodiments, the cancer cells may have a homologous recombination deficiency (HRD). In other embodiments, the cancer cells may have no identified molecular aberrations.

Folate Receptor

In some embodiments, the cancer cells may have elevated expression of the folate receptor (FOLR), especially when associated with aggressively growing cancers. Folate Receptor 1 (FOLR1), also known as Folate Receptor-alpha or Folate Binding Protein, is an N-glycosylated protein expressed on plasma membrane of cells. FOLR1 has a high affinity for folic acid and for several reduced folic acid derivatives. FOLR1 mediates delivery of the physiological folate, 5-methyltetrahydrofolate, to the interior of cells. By way of non-limiting example, FOLR1 is overexpressed in the vast majority of ovarian cancers, as well as in many uterine, endometrial, pancreatic, renal, lung, and breast cancers relative to expression in cells free of cancer, for example on normal tissues such as epithelial cells in the kidney proximal tubules. Other normal tissues such as alveolar pneumocytes, bladder, testes, choroid plexus, and thyroid express FOLR1 and may be used as a control for cancers overexpressing FOLR1.

In some aspects, the methods include detecting the presence of one or more diagnostic or prognostic markers in a sample (e.g. a biological sample from a cancer patient). A variety of screening methods known to one of skill in the art may be used to detect the presence of the marker in the sample including DNA, RNA and protein detection. The techniques can be used to determine the presence or absence or amount of FOLR1 in a sample obtained from a patient relative to a sample obtained from a non-cancer subject. Measurement of FOLR1 in a patient having cancer assists the physician in determining a treatment protocol for the patient. By way of non-limiting example, patients having cancer and having an elevated FOLR1 level relative to a subject without cancer may receive a treatment that includes a therapeutically effective amount of a PARP inhibitor. In some aspects, the level of folic acid may be monitored in the patients receiving PARP inhibitor treatments and a folic acid supplement may be administered.

Currently, there are two principal methods that have been utilized for assessing a subject's “FOLR status”. These include a tissue-based immunohistochemical assay, and a non-invasive radiodiagnostic approach. The latter method is now being tested clinically using 99mTc-EC20. The FOLR status may be measured using an assay such as those provided by/performed by Ventana, HistoGeneX and Phenopath.

Methods of Treatment using a Combination Therapy

In some aspects, methods of combined administration of a PARP inhibitor and a folic acid supplement are provided. Accordingly, the present invention provides a combination therapy for treating or preventing the symptoms of a cancer, comprising administration of a PARP inhibitor and a folic acid supplement. The term “combination therapy”, as used herein, refers to the administration of two or more therapeutic substances, e.g., a PARP inhibitor and a folic acid supplement. The PARP inhibitor may be administered concomitant with, prior to, or following the administration of a folic acid supplement. As set forth herein, a combination therapy involving a PARP inhibitor and a folic acid supplement suppresses the emergence of side effects including anemia to a greater extent than administration of either a PARP inhibitor alone. Standard dosages of the PARP inhibitor and the folic acid supplement are also suitable for the combination therapies described herein. The PARP inhibitor may also be administered with other chemotherapeutic agents and/or radiation therapy.

Accordingly, the invention provides methods of treating cancer in a subject, comprising administering a combination therapy comprising a PARP inhibitor and a folic acid supplement, as described herein. In some embodiments, the method further comprises screening a subject to detect the level of folic acid or the expression level of FOLR1 in the subject.

The PARP inhibitor and the folic acid supplement can be administered to the subject sequentially or simultaneously. A sequential administration includes (a) first administering the PARP inhibitor followed by (b) administering the folic acid supplement. An alternative sequential administration includes (a) first administering the folic acid supplement followed by (b) administering the PARP inhibitor. A simultaneous administration includes administering the PARP inhibitor and the folic acid supplement at the same time; or at substantially the same time.

When administration involves the separate administration (e.g., sequential administration) of the first compound (e.g., a PARP inhibitor) and a second compound (e.g., a folic acid supplement), as described herein, the compounds are administered sufficiently close in time to have the desired therapeutic effect. For example, the period of time between each administration, which can result in the desired therapeutic effect, can range from minutes to hours and can be determined based on the properties of each compound such as potency, solubility, bioavailability, plasma half-life and kinetic profile. For example, the compounds can be administered in any order within about 24 hours of each other or within any time less than 24 hours of each other.

When the PARP inhibitor and the folic acid supplement are administered sequentially, they are separately formulated and can be provided in any order. When the PARP inhibitor and the folic acid supplement are administered simultaneously, however, they may be either separately formulated or combined in the same formulation. When combined in the same formulation, the PARP inhibitor and the folic acid supplement can be formulated so as to be released into the subject at the same time or at different times. The release profile of a formulation comprising both the PARP inhibitor and the folic acid supplement includes the following:

A) release and bioavailability of the PARP inhibitor followed by release and bioavailability of the folic acid supplement;

B) release and bioavailability of the folic acid supplement followed by release and bioavailability of the PARP inhibitor;

C) release and bioavailability of the PARP inhibitor at the same time (or substantially at the same time as) release and bioavailability of the folic acid supplement.

In another embodiment, provided herein is a method of treating cancer in a subject in need thereof comprising administering to the subject a composition comprising a PARP inhibitor and a folic acid supplement, wherein the cancer is selected from the group consisting of breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors, stomach cancer, urinary cancer, and uterine cancer.

A combination of compounds described herein (e.g., a PARP inhibitor and a folic acid supplement) can either result in synergistic increase in anti-cancer activity, or such an increase can be additive. Compositions described herein typically include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to the PARP inhibitor when used alone.

A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the invention provides a pharmaceutical composition of any of the compositions of the present invention. In a related embodiment, the invention provides a pharmaceutical composition of any of the compositions of the present invention and a pharmaceutically acceptable carrier or excipient of any of these compositions.

In one embodiment, the invention includes a packaged cancer treatment. The packaged treatment includes a composition of the invention packaged with instructions for using an effective amount of the composition of the invention for an intended use. In other embodiments, the present invention provides a use of any of the compositions of the invention for manufacture of a medicament to treat a cancer in a subject.

In another embodiment of the invention, a PARP inhibitor and a folic acid supplement can be administered sequentially (in any order) or simultaneously with other pharmaceutical agents typically administered to subjects being treated for cancer. Such other pharmaceutical agents include without limitation anti-emetics, agents that increase appetite, other cytotoxic or chemotherapeutic agents, and agents that relieve pain. The CDA substrate and the compound of any one of formulae I-VIII can be formulated together with or separately from such other pharmaceutical agents.

Pharmaceutical Compositions

The compositions described herein may be used alone or in compositions together with a pharmaceutically acceptable carrier or excipient. Pharmaceutical compositions of the present invention comprise a therapeutically effective amount of a PARP inhibitor and a folic acid supplement, and with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds described herein may be administered to humans and animals in dosage unit formulations containing conventional nontoxic pharmaceutically acceptable carriers, adjuvants, and vehicles as desired.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules or lipid particles, lyophilized powders, or other forms known in the art.

Compositions of the invention may be formulated for delivery as a liquid aerosol or inhalable dry powder. Liquid aerosol formulations may be nebulized predominantly into particle sizes that can be delivered to the terminal and respiratory bronchioles.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono or multi lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1 5 μm. Predominantly in this application means that at least 70% but preferably more than 90% of all generated aerosol particles are within 1 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB® and AERODOSE vibrating porous plate nebulizers (AeroGen, Inc., Sunnyvale, Calif.), SIDESTREAM nebulizers (Medic Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

A compound described herein can be administered alone or in combination with other compounds, for a possible combination therapy being staggered or given independently of one another. Long-term therapy is equally possible as is adjuvant therapy in the con-text of other treatment strategies, as described above. Other possible treatments are therapy to maintain the patient's status after the initial treatment, or even preventive therapy, for example in patients at risk.

Effective amounts of the compounds of the invention generally include any amount sufficient to detectably an inhibition or alleviation of symptoms. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Examples

Severe folic acid deficiency was identified in a patient taking the Poly (ADP-Ribose) Polymerase (PARP)-inhibitor olaparib (Lynparza™) for the treatment of relapsed ovarian cancer. The identification of a nearly undetectable folic acid level without a plausible explanation was intriguing and ultimately led us to identify other patients who also developed severe folic acid deficiency while on olaparib, possibly contributing to anemia and resulting in dose reductions and at times, drug discontinuation. To our knowledge, this is the first description of the association between severe folic acid deficiency and olaparib therapy. We believe that this observation has practical implications such that every effort should be made to identify and treat folate deficiency in patients treated with olaparib. Further exploration of this finding has the potential of providing important insight into olaparib mechanism of action, and interaction between PARP-inhibition, folate pathway, and BRCA mutations in ovarian cancer.

The recommended dose of olaparib is 400 mg twice daily until disease progression or unacceptable toxicity. Olaparib is generally well tolerated. In clinical trials, adverse reactions led to olaparib dose interruptions in 26% of patients, dose reductions in 15% of patients, and discontinuation of olaparib in 9% of patients on olaparib (Olaparib PI).

Olaparib is known to cause hematological toxicity. Reported hematologic side effects include: decreased hemoglobin in 85% to 90% of treated patients (grades ¾: 8% to 15%), increased MCV in 57% to 85% of patients, anemia (25% to 34%; grades ¾: 4% to 18%), decreased neutrophils (25% to 32%; grades ¾: 7% to 8%), decreased platelet count (26% to 30%; grades ¾: 3% to 6%) [ref; package insert]. Furthermore, myelodysplastic syndrome/Acute Myeloid Leukemia were reported in 2% of olaparib-treated patients (Olaparib PI). The manufacturer's recommendations for the management of hematologic toxicity involve monitoring blood counts at baseline and monthly thereafter, and interrupting treatment until any hematologic toxicity has resolved to s grade 1, with consideration for additional evaluation if count recovery does not take place after 4 weeks of treatment interruption, including bone marrow and cytogenetic analyses [ref, drug insert]. Notably, little is known about the etiology of olaparib-induced hematological toxicity. Herein we describe the first report and subsequent case series of severe folic acid deficiency induced by olaparib therapy.

Case Report

A 61 year-old woman with type 2 diabetes mellitus, chronic heart failure, atrial fibrillation, hypertension, morbid obesity s/p gastric bypass, obstructive sleep apnea, and depression/anxiety was diagnosed with Stage IIIC poorly differentiated papillary serous ovarian cancer in 2013. She underwent TAH/BSO and suboptimal debulking surgery followed by 6 cycles of adjuvant carboplatin and paclitaxel, and avastin maintenance. In May of 2016, a re-staging CT scan demonstrated disease progression. She received one cycle of chemotherapy with carboplatin/gemcitabine, which was poorly tolerated. In August 2016, she was started on olaparib based on a positive homologous recombination deficiency (HRD) assay performed on her tumor. After 5 weeks of therapy she was noted to have severe anemia (hgb 6.6 g/dL) requiring blood transfusion. Olaparib was dose-reduced to 200 mg BID and one week later it was reduced further to 100 mg BID due to persistent anemia. She was referred to Hematology for the evaluation of macrocytic anemia. At the time of Hematology evaluation, her CBC was remarkable for macrocytic anemia (Hgb 9.0 g/dL, MCV 110.7 fL). Other parameters including platelet, white cell counts, iron studies and Vitamin B12 level were normal. However, she had an exceedingly low folate level reported at <1.6 ng/mL. (Normal: 7-31.4 ng/mL). Notably, chart review was remarkable for long-standing anemia which had worsened since the initiation of olaparib. In 2016, 3 months prior to starting Olaparib, she was hospitalized after carboplatin/gemcitabine chemotherapy and was noted to have macrocytic anemia, (hgb of 8.1 g/dL, MCV 104 fL) but her folic acid level was normal at 14 ng/ml. She was promptly started on oral folic acid supplementation at 1 mg daily. Subsequently, her anemia and macrocytosis have improved. She has not required any further blood transfusions. She continued on olaparib and tolerated dose escalation without further complications. We then sought to determine whether folic acid deficiency existed in other olaparib-treated patients, and identified 3 other women with folic acid level significant reduction in their folate level reported at <1.6 ng/mL as shown in Table 1.

TABLE 1
Clinical characteristics and serum folate level in ovarian cancer patients
with severe anemia on olaparib

| Patient/ | | | | |
|---|---|---|---|---|
| Age | A/61 | B/78 | C/63 | D/57 |
| Diagnosis | HRD⁶ | HRD⁶ | HRD⁶ | Recurrent |
| | positive | positive | positive | ovarian |
| | recurrent | recurrent | recurrent | cancer; |
| | ovarian | ovarian | ovarian | RAD51C |
| | cancer | cancer | cancer | positivity |
| Days of | 131 | 155 | 136 | 361 |
| Olaparib Use | | | | |
| Baseline | 8.5/ | 8.4/ | 10.4/ | 11.8/ |
| Hgb/MCV¹ | 103.6 | 100.0 | 98.4 | 97.0 |
| Hgb/MCV | 6.6/ | 6.3/ | 6.3/ | 8.8/ |
| Nadir¹ | 110.7 | 102.0 | 91.6* | 98.1 |
| Days to Hgb | 36 | 26 | 55 | 151 |
| Nadir¹ | | | | |
| Folic Acid | <1.6 | <1.6 | <1.6 | 1.6 |
| Level² | | | | |
| Other | Leukopenia | Leukopenia | None | Leukopenia |
| Hematologic | Thrombo- | Thrombo- | | Thrombo- |
| Abnormalities³ | cytopenia | cytopenia | | cytopenia |
| Transfusion | Yes | Yes | No | No |
| requirement⁴ | | | | |
| Dose Reduction | Yes/ | Yes/ | Yes/ | No |
| or Delay of | anemia | anemia | anemia | |
| Olaparib/Cause | | | | |
| Response to | Yes | Yes | Yes | Yes |
| folic acid | | | | |
| supplemen- | | | | |
| tation⁵ | | | | |

¹Hemoglobin measured in g/dL (reference range 12.0-16.0 g/dL), MCV measured in fL (reference range 82.0-103.0 fL)
²Folic Acid level measured in ng/mL (reference range 7.0-31.4 ng/mL)
³Other hematologic abnormalities noted at time of initial Hematology assessment
⁴Transfusion requirement secondary to anemia noted after administration of Olaparib
⁵Response to folic acid supplementation measured as improvement of folic acid level to within the reference range (≥7.0 ng/mL)
⁶HRD = Homologous recombination deficiency
*Notably, hemoglobin nadir reached after initiation of folic acid supplementation in this patient.

Methods

A formal assessment of folic acid level along with a chart review of olaparib treated ovarian cancer patients was conducted to determine the frequency of severe folic acid deficiency in this population and the impact of replacement therapy on outcome. This was an IRB-approved study performed at Rush University Medical Center from Dec. 1, 2016 to Apr. 3, 2017. Data collection was conducted in accordance with good clinical practice guidelines and the provisions of declaration of Helsinki. Data collected included cancer history, date of initiation of olaparib, duration of olaparib use, CBC data, peripheral smear/RBC morphology data, Vitamin B12, folic acid levels, iron studies with ferritin, reticulocyte count, LDH levels, haptoglobin levels, TSH levels, any treatment administered for hematological toxicities including nutritional supplementation or blood product transfusion, dose reductions or cessation of olaparib use and indication for such intervention were collected for all patients.

Results

Three additional patients were identified who developed severe folic acid deficiency (<1.6 ng/mL) after taking olaparib for at least four weeks. Patient A was previously described in the Case Report above. Patient B developed an acute decrease in Hgb (8 g/dL to 6.8 g/dL) requiring blood transfusion five weeks after starting olaparib. Olaparib was subsequently held. She was found to have severe folic acid deficiency (<1.6 ng/mL). Two weeks after starting folic acid 1 mg daily her Hgb markedly improved and she has no longer required blood transfusions. Olaparib was restarted and she is tolerating therapy well. Patient C was noted to have a drop in baseline Hgb (14 g/dL to 10 g/dL) approximately 4 weeks after starting olaparib, as well as severe folic acid deficiency (<1.6 ng/mL). At that time she was started on folic acid 1 mg daily. Two weeks later her labs showed an even further decrease in Hgb (6.3 g/dL) requiring blood transfusion. Folic acid was minimally increased (2.9 ng/mL) from prior value. Olaparib was briefly held. Her folic acid supplementation was increased to 2 mg TID. Her Hgb stabilized at around 9 g/dL and folic acid level normalized. She was restarted on Olaparib and continues to tolerate the medication. Patient D developed mild anemia while on olaparib for 6 months (ranging 9-10 g/dL). Workup of anemia revealed a severely low folic acid level (1.6 ng/mL). She was started on folic acid 1 mg daily with subsequent improvement in her folic acid level and remains on therapeutic dose of olaparib with stable disease.

### DISCUSSION

In this report, we present 4 cases of severe folate deficiency in the setting of olaparib therapy for ovarian CA. Three of the four patients we discuss developed transfusion-dependent anemia in the course of their treatment with olaparib (Table 1). Available laboratory data suggest that folate level drops precipitously within weeks after initiation of olaparib. We further demonstrated that folic acid supplementation albeit at variable doses can correct olaparib-induced folic acid deficiency.

We theorize that olaparib may have an off-target effect thereby inducing folate deficiency. We are not aware of other reports describing this association. As such, monitoring and identification of folate deficiency may abrogate the need for dose reduction/modification if treated in a timely manner. As folate deficiency can cause dysplastic changes in the bone marrow, this may result in an erroneous diagnosis of MDS. Identifying and treating folate deficiency may improve the efficacy, safety, and cost-effectiveness of olaparib.

It is certainly conceivable, that olaparib and other PARP-inhibitors at least partially exert their anti-neoplastic effect by interfering with folate pathway. Folic acid is a vitamin which is required for DNA synthesis and repair. Anti-folates (and specifically, methotrexate) are one of the first anti-neoplastic agents developed in 1940s by Sidney Farber, MD who was credited for this discovery as the “father of chemotherapy.” Methotrexate is known to cause variably decreased serum folate level (Hellman, 1964). Recent research identified the abundance of folate receptor-α (FR-α) in many solid tumors with the high prevalence in ovarian cancer (Bergamini A, 2016). This observation made FR-α a very attractive target for novel anti-neoplastic agents (Assaraf YG, 2014). Several anti-folate agents including monoclonal antibodies to FR-α are in clinical trials for ovarian cancer as well as other malignancies (Bergamini A, 2016). As the structure of studied PARP-inhibitors resembles the structure of folic acid, it is possible that they are able to competitively bind to folate receptors in the gut and the tumor leading to impaired folate absorption and anemia as well as decreased cancer cell proliferation.

Hematologic toxicity has been observed with all studied PARP-inhibitors and is a known class side effect. However, further studies must be performed to identify whether folic acid deficiency is the causative etiology of this association. If demonstrated, one possible explanation lies in the chemical structure and mechanism of action of PARP-inhibitors. Olaparib, rucaparib, and other most studied PARP-inhibitors belong to NAD (nicotinamide adenine dinucleotide) mimetics (Thomas, 2016; Konecny, 2016). However, NAD pathway is known to interact with the folate pathway (Locasale, 2013). If this explanation is correct, then, non-NAD agonistic PARP inhibitors which are currently under development (Thomas, 2016) may not cause folate deficiency.

There is no known association between the folate and BRCA pathways, and no difference in the incidence of hematologic side effects in gBRCA carriers vs non-carriers in PARP-inhibitor clinical trials. Interestingly, none of 4 patients reported here had a germline BRCA mutation. The question as to whether decreased folate level induced by olaparib is required to exert the therapeutic efficacy of olaparib, and whether folic acid supplementation may be deleterious to this effect needs to be answered in a prospective fashion.

Conclusions:

Anemia in olaparib treated patients may be the result of severe folate deficiency induced by the drug. Our study suggests that testing for, and correction of folic acid deficiency may be beneficial in patients who are receiving treatment with olaparib. Further studies are necessary to confirm this association and elucidate the mechanism of folic acid deficiency during PARP inhibitor therapy in advanced ovarian cancer.

Tables 2a, 2b, 3a and 3b include the clinical characteristics and serum folate level in ovarian cancer patients including the four patients described above as well as additional patients that were screened.

TABLE 2a
Clinical characteristics and serum folate level in ovarian cancer
patients with severe anemia on olaparib

| Patient/Age | A/61 | B/78 | C/63 | D/57 |
|---|---|---|---|---|
| Diagnosis | HRD⁶ | HRD positive | HRD | Recurrent |
| | positive | recurrent ovarian | positive | ovarian |
| | recurrent | cancer, early | recurrent | cancer |
| | ovarian | stage breast | ovarian | with |
| | cancer | cancer, early | cancer | RAD51 |
| | | stage NSCLC | | positivity |
| Days of | 171 | 92 | 73 | 298 |
| Olaparib Use | | | | |
| Baseline | 8.5/ | 8.4/ | 10.4/ | 11.8/ |
| Hgb/MCV¹ | 103.6 | 100.0 | 98.4 | 97.0 |
| Hgb/MCV | 6.6/ | 6.3/ | 6.3/ | 8.8/ |
| Nadir¹ | 110.7 | 102.0 | 91.6* | 98.1 |
| Days to Hgb | 36 | 26 | 55 | 151 |
| Nadir¹ | | | | |
| Folic Acid | <1.6 | <1.6 | <1.6 | 1.6 |
| Level² | | | | |
| Other | Leukopenia; | Leukopenia; | None | Leukopenia; |
| Hematologic | Thrombo- | Thrombo- | | Thrombo- |
| Abnormalities³ | cytopenia | cytopenia | | cytopenia |
| Transfusion | Yes | Yes | No | No |
| requirement⁴ | | | | |
| Dose | Yes, anemia | Yes, anemia | Yes, | No |
| Reduction | | | anemia | |
| or Delay of | | | | |
| Olaparib/ | | | | |
| Cause | | | | |
| Response to | Yes | Yes | No | Not yet |
| folic acid | | | | assessed |
| supplemen- | | | | |
| tation⁵ | | | | |

¹Hemoglobin measured in g/dL (reference range 12.0-16.0 g/dL), MCV measured in fL (reference range 82.0-103.0 fL)
²Folic Acid level measured in ng/mL (reference range 7.0-31.4 ng/mL)
³Other hematologic abnormalities noted at time of initial Hematology assessment
⁴Transfusion requirement secondary to anemia noted after administration of Olaparib
⁵Response to folic acid supplementation measured as improvement of folic acid level to within the reference range (≥7.0 ng/mL)
⁶HRD = Homologous recombination deficiency
*Notably, hemoglobin nadir reached after initiation of folic acid supplementation in this patient
**Patient with no germline mutations, no HRD mutations (olaparib off-label use).

TABLE 2b
Clinical characteristics and serum folate level in ovarian cancer
patients with severe anemia on olaparib

| Patient/Age | E/41 | F/72 | G/94 | H/55 |
|---|---|---|---|---|
| Diagnosis | Recurrent ovarian | BRCA2 mutated | Recurrent | BRCA1 |
| | cancer | recurrent ovarian | ovarian | mutated |
| | with RAD51 | cancer | cancer** | recurrent |
| | positivity | | | |
| Days of | 90 | 427 | 102 | 357 |
| Olaparib Use | | | | |
| Baseline | 12.2/ | 11.1/ | 9.9/ 93.6 | 8.8/ |
| Hgb/MCV¹ | 111.8 | 103.4 | | 98.5 |
| Hgb/MCV Nadir¹ | 11.2/ | 5.0/ | 8.4/ | 5.8/ |
| | 112.0 | 115.8 | 93.2 | 98.9 |
| Days to Hgb Nadir¹ | 5 | 167 | 41 | 58 |
| Folic Acid Level² | 4.9 | 5.2 | 3.7 | 6.2 |
| Other Hematologic | Leukopenia; | Leukopenia | Leukopenia | Leukopenia; |
| Abnormalities³ | Thrombocytopenia | | | Thrombocytopenia |
| Transfusion | No | Yes | No | Yes |
| requirement⁴ | | | | |
| Dose Reduction or | No | Yes, neutropenia, | Yes, folic acid | No |
| Delay of Olaparib/ | | anemia, fatigue, | deficiency | |
| Cause | | dyspnea | | |
| Response to folic | Not yet assessed | Not yet assessed | Yes | Not yet assessed |
| acid | | | | |
| supplementation⁵ | | | | |

¹Hemoglobin measured in g/dL (reference range 12.0-16.0 g/dL), MCV measured in fL (reference range 82.0-103.0 fL)
²Folic Acid level measured in ng/mL (reference range 7.0-31.4 ng/mL)
³Other hematologic abnormalities noted at time of initial Hematology assessment
⁴Transfusion requirement secondary to anemia noted after administration of Olaparib
⁵Response to folic acid supplementation measured as improvement of folic acid level to within the reference range (≥7.0 ng/mL)
⁶HRD = Homologous recombination deficiency
*Notably, hemoglobin nadir reached after initiation of folic acid supplementation in this patient
**Patient with no germline mutations, no HRD mutations (olaparib off-label use).

TABLE 3a
Clinical characteristics and serum folate level in ovarian cancer
patients with severe anemia on olaparib

| Patient/Age | A/62 | B/78 | C/63 | D/57 |
|---|---|---|---|---|
| Diagnosis | HRD-positive | HRD-positive | HRD-positive | Recurrent ovarian |
| | recurrent | recurrent | recurrent | cancer; germ line |
| | ovarian | ovarian | ovarian | mutation in |
| | cancerⁱ | cancerⁱ | cancerⁱ | RAD51C gene |
| Days of Olaparib | 192 | 201 | 208 | 367 |
| Use | | | | |
| Hgb^{b} Baseline^{g}/ | 8.5/ | 8.4/ | 10.4/ | 11.8/ |
| MCV^{a} Baseline^{g} | 103.6 | 100.0 | 98.4 | 97.0 |
| Hgb^{b} Nadir^{h}/ | 6.6/ | 6.3/ | 6.3/ | 8.8/ |
| MCV^{a} Nadir^{h} | 110.7 | 102.0 | 91.6 | 98.1 |
| Days to Hgb | 36 | 26 | 55 | 151 |
| Nadir¹ | | | | |
| Folic Acid Level^{c} | 14/<1.6 | <1.6 | 1.6 | <1.6 |
| Baseline^{g}/ | | | | |
| Folic Acid Level^{c} | | | | |
| Nadir^{h} | | | | |
| Other | Thrombo- | Leukopenia; | None | Leukopenia; |
| Hematologic | cytopenia | Thrombo- | | Thrombo- |
| Abnormalities^{d} | | cytopenia | | cytopenia |
| Transfusion | Yes | Yes | Yes | No |
| required^{e} | | | | |
| Dose Reduction | Yes/anemia | Yes/anemia | Yes/anemia | No |
| or Delay of | | | | |
| Olaparib/Cause | | | | |
| Response to folic | Yes | Yes | Yes | Yes |
| acid | | | | |
| supplementation^{f} | | | | |
| Treatment | Treatment for 6 | Treatment for 6 | Treatment for 7 | Treatment for 12 |
| outcome | months; | months; | months; | months; |
| | discontinued | discontinued | discontinued | discontinued |
| | because of | because of | because of | because of severe |
| | progression | refractory anemia | progression | diarrhea |

Abbreviations: HgB: hemoglobin; HRD: homologous recombination deficiency; MCV, mean corpuscular volume.
^{a}MCV reference range, 82.0 to 103.0 fL.
^{b}HgB reference range, 12.0 to 16.0 g/dL.
^{c}Folic acid reference range, 7.0 to 31.4 ng/mL.
^{d}Noted at time of initial hematologic assessment.
^{e}Describes transfusion requirement secondary to anemia noted while patient receiving olaparib.
^{f}Defined as improvement of folic acid level to within reference range.
^{g}Defined as no more than 14 days before olaparib treatment initiation.
^{h}Defined as lowest value documented while patient was receiving olaparib.
ⁱHRD assay used: myChoice,; Myriad Genetics Laboratories, Salt Lake City, UT
^{k}Notably, this patient was administered a reduced dose of olaparib from the onset (200 mg once per day) because of poor functional status.

TABLE 3b
Clinical characteristics and serum folate level in ovarian cancer
patients with severe anemia on olaparib

| Patient/Age | E/73 | F/95 | G/52 | H/35 |
|---|---|---|---|---|
| Diagnosis | Germ line | Recurrent ovarian | Recurrent ovarian | Metastatic breast |
| | BRCA2-mutated | cancer; off-label | cancer; germ line | cancer; germ line |
| | recurrent | use (no germ line | mutation in | mutation in BRIP1 |
| | ovarian | or somatic BRCA | BRCA1 gene | |
| | cancer | mutations | | |
| | | demonstrated)^{j} | | |
| Days of Olaparib | 20 | 133 | 559 | 84 |
| Use | | | | |
| Hgb^{b} Baseline^{g}/ | 11.4/ | 10.2/ | 12.1/ | 11.9/ |
| MCV^{a} Baseline^{g} | 108 | 94.7 | 93.8 | 84.6 |
| Hgb^{b} Nadir^{h}/ | 10.8/ | 8.1/ | 12.2/ | 10.4/ |
| MCV^{a} Nadir^{h} | 105.6 | 103.1 | 93.5 | 85.8 |
| Days to Hgb | 19 | 129 | 37 | 63 |
| Nadir¹ | | | | |
| Folic Acid Level^{c} | <1.6 | 3.7 | 9.8 | <1.6 |
| Baseline^{g}/ | | | | |
| Folic Acid Level^{c} | | | | |
| Nadir^{h} | | | | |
| Other | Leukopenia; | Leukopenia; | None | None |
| Hematologic | Thrombo- | T | | |
| Abnormalities^{d} | cytopenia | | | |
| Transfusion | No | No | No | No |
| required^{e} | | | | |
| Dose Reduction | No | No^{k} | Yes/rash | No |
| or Delay of | | | | |
| Olaparib/Cause | | | | |
| Response to folic | No | Yes | No | No |
| acid | supplementation | | supplementation | supplementation |
| supplementation^{f} | | | | |
| Treatment | Treatment stopped | Treatment for 4 | Treatment for 18 | Treatment for 3 |
| outcome | after 20 days | months; | months; ongoing | months; |
| | because of | discontinued | | discontinued |
| | progression | because of | | because of |
| | | progression | | progression |

Abbreviations: HgB: hemoglobin; HRD: homologous recombination deficiency; MCV, mean corpuscular volume.
^{a}MCV reference range, 82.0 to 103.0 fL.
^{b}HgB reference range, 12.0 to 16.0 g/dL.
^{c}Folic acid reference range, 7.0 to 31.4 ng/mL.
^{d}Noted at time of initial hematologic assessment.
^{e}Describes transfusion requirement secondary to anemia noted while patient receiving olaparib.
^{f}Defined as improvement of folic acid level to within reference range.
^{g}Defined as no more than 14 days before olaparib treatment initiation.
^{h}Defined as lowest value documented while patient was receiving olaparib.
ⁱHRD assay used: myChoice,; Myriad Genetics Laboratories, Salt Lake City, UT
^{k}Notably, this patient was administered a reduced dose of olaparib from the onset (200 mg once per day) because of poor functional status.

### Additional Examples

Patients receiving PARP inhibitor treatment for cancers including but not limited to, breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors, stomach cancer, urinary cancer, and uterine cancer, will also be evaluated for folic acid deficiency using the methods described above. The patients with folic acid deficiency will receive subsequent combination treatment with a PARP inhibitor and a folic acid supplement. Data to be collected will include cancer history, date of initiation of the PARP inhibitor, duration of the PARP inhibitor use, CBC data, peripheral smear/RBC morphology data, Vitamin B12, folic acid levels, iron studies with ferritin, reticulocyte count, LDH levels, haptoglobin levels, TSH levels, any treatment administered for hematological toxicities including nutritional supplementation or blood product transfusion, dose reductions or cessation of the PARP inhibitor use and indication for such intervention were collected for all patients.

Patients receiving PARP inhibitor treatment for cancer will be evaluated for folic acid deficiency using the method described above. The PARP inhibitors to be evaluated are selected from, but not limited to the following: olaparib, rucaparib, niraparib, talazoparib, veliparib, CEP8983, CEP9722, E7016, INO-1001, AZD2461, E7449, SC10914, BGB-290 and fluzoparib. The patients with folic acid deficiency will receive subsequent combination treatment with a PARP inhibitor and a folic acid supplement. Data to be collected will include cancer history, date of initiation of the PARP inhibitor, duration of the PARP inhibitor use, CBC data, peripheral smear/RBC morphology data, Vitamin B12, folic acid levels, iron studies with ferritin, reticulocyte count, LDH levels, haptoglobin levels, TSH levels, any treatment administered for hematological toxicities including nutritional supplementation or blood product transfusion, dose reductions or cessation of the PARP inhibitor use and indication for such intervention were collected for all patients.

Patients having cancer will be evaluated for folate receptor expression levels. Patients having elevated folate receptor expression levels relative to a control subject without cancer will receive a therapeutically effective amount of a PARP inhibitor. Patients having an elevated folate receptor expression level and receiving a therapeutically effective amount of a PARP inhibitor may also be monitored for a folic acid deficiency. Patients having a folic acid deficiency will receive a folic acid supplement.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### REFERENCES

Assaraf. Y. G., Leamon, C. P., and Reddy, J. A. (2014) The folate receptor as a rationale therapeutic target for personalized cancer treatment. Drug Resist Updat., 17(4-6):89-95.
Bergamini, A., Ferrero, S., Leone Roberti Maggiore, U., Scala, C., Pella, F., Vellone, V. G., Petrone, M., Rabaiotti, E., Cioffi, R., Candiani, M., and Mangili, G. (2016) Folate receptor alpha antagonists in preclinical and early stage clinical development for the treatment of epithelial ovarian cancer. Expert Opin Investig Drugs., 25(12):1405-1412.
Hellman, S., lannotti, A. T., and Bertino, J. R. (1964) Determinations of the levels of serum folate in patients with carcinoma of the head and neck treated with methotrexate. Cancer Res., 24:105-13.
Konecny, G. E., and Kristeleit, R. S. (2016) PARP inhibitors for BRCA1/2-mutated and sporadic ovarian cancer: current practice and future directions. Br J Cancer., 115(10):1157-1173.
Ledermann, J., Harter, P., Gourley, C., Friedlander, M., Vergote, I., Rustin, G., Scott, C., Meier, W., Shapira-Frommer, R., Safra, T., Matei, D., Fielding, A., Spencer, S., Dougherty, B., Orr, M., Hodgson, D., Barrett, J., and Matulonis, U. (2014) Olaparib maintenance therapy in patients with platinum-sensitive relapsed serous ovarian cancer: a preplanned retrospective analysis of outcomes by BRCA status in a randomized phase 2 trial. Lancet Oncol., 15(8), 852-861.
Ledermann, J., Harter, P., Gourley, C., Friedlander, M., Vergote, I., Rustin, G., Scott, C., Meier, W., Shapira-Frommer, R., Safra, T., Matei, D., Fielding, A., Spencer, S., Rowe, P., Lowe, E., Hodgson, D., Sovak, M. A., and Matulonis, U. (2016) Overall survival in patients with platinum-sensitive recurrent serous ovarian cancer receiving olaparib maintenance monotherapy: an updated analysis from a randomised, placebo-controlled, double-blind, phase 2 trial. Lancet Oncol., 17(11), 1579.
Lheureux, S., Bruce, J. P., Burnier, J. V., Karakasis, K., Shaw, P. A., Clarke, B. A., Yang, S. Y., Quevedo, R., Li, T., Dowar, M., Bowering, V., Pugh, T. J., and Oza, A. M. (2017) Somatic BRCA1/2 recovery as a resistance mechanism after exceptional response to poly(ADP-ribose) polymerase inhibition. J Clin Oncol., 25(11): 1240-1249.
Locasale, J. W. (2013) Serine, glycine and the one-carbon cycle: cancer metabolism in full circle. Nat Rev Cancer., 13(8): 572-583.
Spriggs, D. R., and Longo, D. L. (2016) PARP inhibitors in ovarian cancer treatment. N Engl J Med., 375(22):2197-2198.
Thomas, C., Ji, Y., Lodhi, N., Kotova, E., Pinnola, A. D., Golovine, K., Makhov, P., Pechenkina, K., Kolenko, V., and Tulin, A. V. (2016) Non-NAD-Like poly(ADP-Ribose) Polymerase-1 inhibitors effectively eliminate cancer in vivo. EBioMedicine, https: doi: 10.1016/j. ebiom.2016.10.001.

1. A method of treating cancer in a subject comprising:
administering a therapeutically effective amount of a poly (ADP ribose) polymerase (PARP) inhibitor to the subject in need thereof; and
administering a therapeutically effective amount of a folic acid supplement to the subject.

2. The method according to claim 1, wherein the PARP inhibitor is selected from the group consisting of olaparib, rucaparib, niraparib, talazoparib, veliparib, CEP8983, CEP9722, E7016, INO-1001, AZD2461, E7449, SC10914, BGB-290 or fluzoparib.

3. The method according to claim 1, wherein the PARP inhibitor is olaparib.

4. The method according to claim 1, wherein the cancer is selected from the group consisting of breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors stomach cancer, urinary cancer, or uterine cancer.

5. The method according to claim 1, wherein the subject has a BRCA1 or BRCA2 mutation.

6. The method according to claim 1, further comprising monitoring folic acid levels in the subject.

7. The method according to claim 1, wherein the amount of the folic acid supplement is 0.25 mg/day up to 6 mg/day.

8. The method according to claim 1, wherein the PARP inhibitor is co-administered with a chemotherapeutic agent or radiotherapy.

9. A method of treating cancer in a subject comprising:
identifying an elevated folate receptor expression level on cancer cells of the subject relative to a folate receptor expression level on cells in a subject free of cancer; and
administering a therapeutically effective amount of a PARP inhibitor to the subject having the elevated folate receptor level.

10. The method according to claim 9, further comprising monitoring a folic acid level in the subject.

11. The method according to claim 9, further comprising administering a therapeutically effective amount of a folic acid supplement to the subject.

12. The method according to claim 9, wherein the amount of the folic acid supplement is 0.25 mg/day up to 6 mg/day.

13. The method according to claim 9, wherein the cancer is selected from the group consisting of breast cancer, cervical cancer, choriocarcinoma, colorectal cancer, endometrial cancer, endocrine cancer, esophageal cancer, fallopian tube cancer, glioblastoma, gastric cancer, gastrointestinal cancer, head cancer, hematological malignancies, including B-cell malignancies, leukemia/lymphoma, liver cancer, lung cancer, melanoma, neck cancer, ovarian cancer, pancreatic cancer, peritoneal cancer, prostate cancer, skin cancer, solid tumors stomach cancer, urinary cancer, or uterine cancer.

14. The method according to claim 9, wherein the PARP inhibitor is selected from the group consisting of olaparib, rucaparib, niraparib, talazoparib, veliparib, CEP8983, CEP9722, E7016, INO-1001, AZD2461, E7449, SC10914, BGB-290 or fluzoparib.

15. A composition for the treatment of cancer, the composition comprising:
a PARP inhibitor and a folic acid supplement.

16. The composition according to claim 15, wherein the PARP inhibitor is selected from the group consisting of olaparib, rucaparib, niraparib, talazoparib, veliparib, CEP8983, CEP9722, E7016, INO-1001, AZD2461, E7449, SC10914, BGB-290 or fluzoparib.

17. A method of identifying a subject having cancer that is likely to benefit from a PARP inhibitor therapy, the method comprising:
identifying an elevated folate receptor expression level on cancer cells of the subject relative to a folate receptor expression level on cells in a subject free of cancer; wherein subjects having the elevated folate receptor expression level on cancer cells are likely to benefit from a PARP inhibitor therapy.

18. The method according to claim 17, further comprising administering a therapeutically effective amount of a PARP inhibitor to the subject having the elevated folate receptor level.
